(19) United States
(12) Patent Application Publication
(10) Pub. No.: US 2026/0174922 A1
(43) Pub. Date: Jun. 25, 2026

(54) AIR DEODORIZING AND DISINFECTING SYSTEM

(71) Applicants: Abu Ahmed Majumder, Torrance, CA (US); Millie Majumder, Torrance, CA (US); Adiba Majumder, Torrance, CA (US)

(72) Inventors: Abu Ahmed Majumder, Torrance, CA (US); Millie Majumder, Torrance, CA (US); Adiba Majumder, Torrance, CA (US)

(21) Appl. No.: 18/987,857

(22) Filed: Dec. 19, 2024

(51) Int. Cl. A61L 9/12

(52) U.S. Cl. CPC A61L 9/12; A61L 2209/15

(57) ABSTRACT

An air treatment system for deodorizing and disinfecting air includes a tablet comprising a volatile substance such that the tablet readily vaporizes. The volatile substance comprises one or more materials configured to treat a surrounding atmosphere such as by deodorizing the surrounding atmosphere or disinfecting the surrounding atmosphere. A container is provided for housing the tablet and comprises a cup which is air permeable to allow air to flow through the cup and over the tablet. An adhesive is coupled to the cup and may be used to mount the cup to a support surface such as a vertical wall surface.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to air treatment apparatuses and more particularly pertains to a new air treatment apparatus for deodorizing and disinfecting air.

(2) Description of Related Art Including Information Disclosed under 37 CFR 1.97 and 1.98.

The prior art describes various air treatment apparatuses which deodorize or disinfect the air via volatile substances that vaporize readily into the air. However, the prior art fails to describe such an apparatus in which a container is provided for holding one or more tablets of the volatile substances, facilitating airflow through the container, and mounting to a support surface such as a vertical wall surface.

### (h) BRIEF SUMMARY OF THE INVENTION

An embodiment of the disclosure meets the needs presented above by generally comprising a tablet which includes a volatile substance such that the tablet readily vaporizes. The volatile substance comprises one or more materials configured to treat a surrounding atmosphere such as by deodorizing the surrounding atmosphere or disinfecting the surrounding atmosphere. A container is provided for housing the tablet and comprises a cup which includes a base wall and a perimeter wall. The perimeter wall is coupled to and extends upwardly from the base wall, and the container defines an interior space surrounded by the base wall and the perimeter wall. An upper edge of the perimeter wall defines an access opening to the interior space. The cup defines a plurality of airflow apertures which extend through the cup into the interior space to facilitate airflow through the cup. The cup further comprises a mount member which is coupled to and extends outwardly from the perimeter wall. The mount member has a mount surface which faces away from the perimeter wall. An adhesive is coupled to the mount surface of the mount member and is configured to secure the container to a support surface.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of an air treatment system according to an embodiment of the disclosure.

FIG. 2 is a bottom perspective view of an embodiment of the disclosure.

FIG. 3 is a top perspective view of a cup and a plurality of tablets of an embodiment of the disclosure.

FIG. 4 is an in-use view of an embodiment of the disclosure.

FIG. 5 is a cross-sectional view of an embodiment of the disclosure taken from Line 5-5 in FIG. 4.

### (j) DETAILED DESCRIPTION OF THE INVENTION

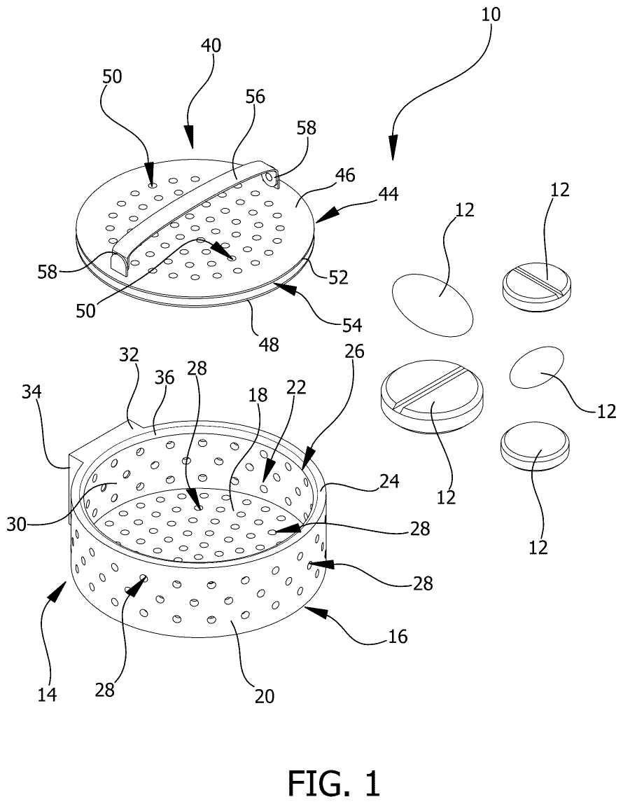

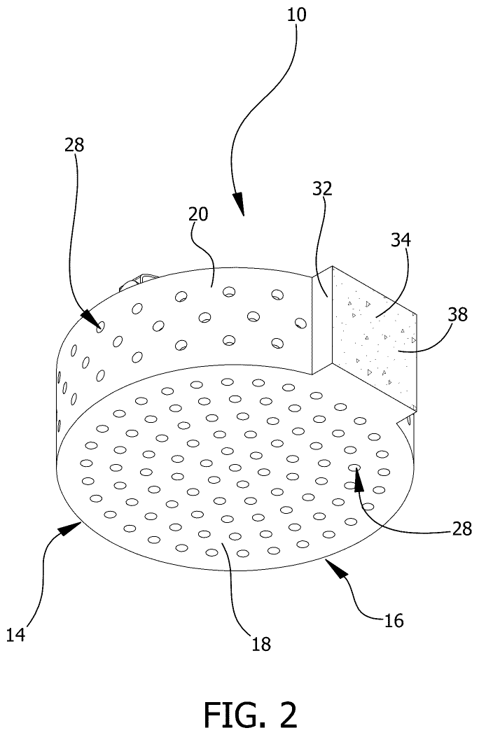

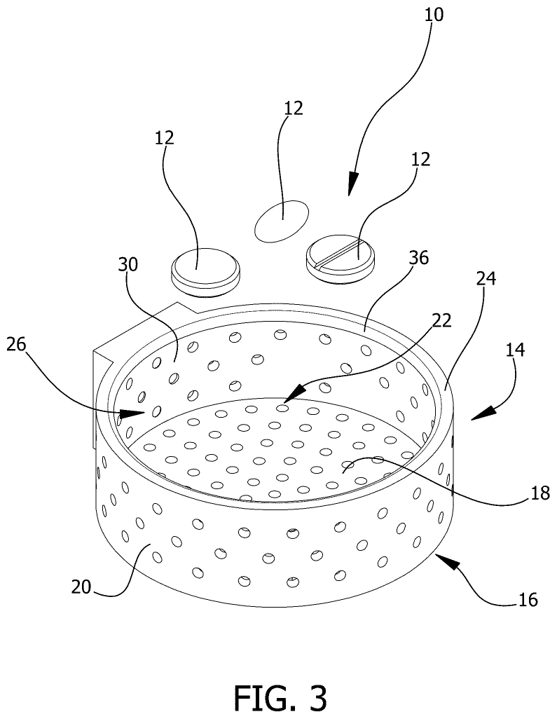

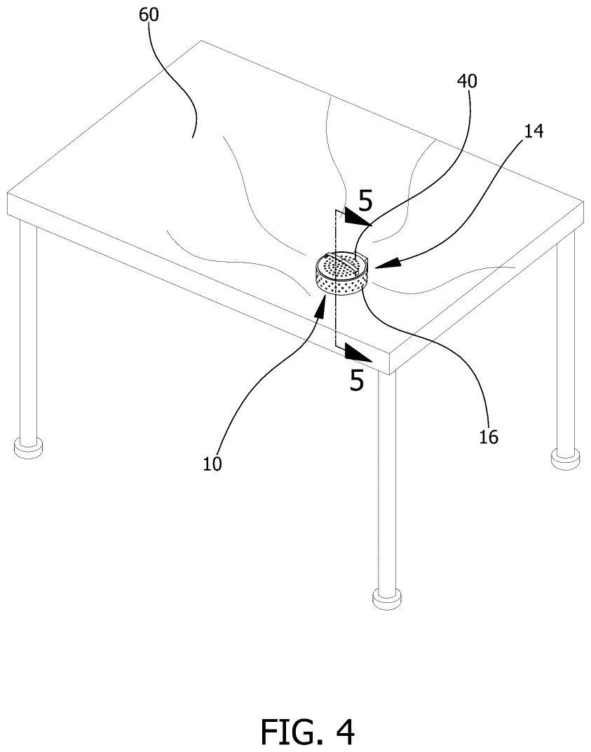

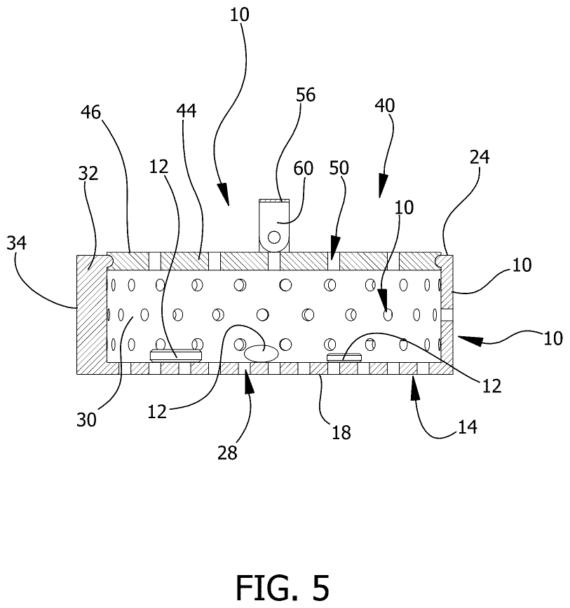

With reference now to the drawings, and in particular to FIGS. 1 through 5 thereof, a new air treatment apparatus embodying the principles and concepts of an embodiment of the disclosure and generally designated by the reference numeral 10 will be described.

As best illustrated in FIGS. 1 through 5, the air treatment system 10 generally comprises a plurality of tablets 12 each of which comprises a plurality of volatile substances wherein the tablet 12 readily vaporizes. The plurality of volatile substances includes a deodorizing material configured to deodorize a surrounding atmosphere and a disinfecting material configured to disinfect the surrounding atmosphere such as through an antimicrobial property. The disinfecting material may, for example, comprise hydrogen peroxide or a like material. Para-dichlorobenzene may be used to compose, at least in part, the deodorizing material, the disinfecting material, or both. The deodorizing material may include a fragrant material to mask odors and may include one or more solvents, emulsifiers, and surfactants to dissolve or emulsify odor-causing substances. For example, the deodorizing material may include ethanol, mineral oil, or glycol ethers. Other substances may be included in the tablets 12 to promote vaporization of the deodorizing material or the disinfecting material, for preservative purposes, to impart a color or texture to the tablets 12, or other uses may also be incorporated into the tablets 12. The tablets 12 may vary in shape and may have circular disc shapes, oval prism shapes, prolate spheroid shapes (i.e., football-like), spherical shapes, and the like.

A container 14 for housing the plurality of tablets 12 is provided which comprises a cup 16, an adhesive 38, and a lid 40. The cup 16 comprises a base wall 18 and a perimeter wall 20, where the perimeter wall 20 is coupled to and extends upwardly from the base wall 18. The container 14 defines an interior space 22 surrounded by the base wall 18 and the perimeter wall 20. An upper edge 24 of the perimeter wall 20 defines an access opening 26 to the interior space 22. The cup 16 also defines a plurality of airflow apertures 28 which extend through the cup 16 into the interior space 22 to facilitate airflow through the cup 16. The plurality of airflow apertures 28 is distributed across the base wall 18 and the perimeter wall 20. The perimeter wall 20 has a cylindrical shape but may have a prismatic shape or another suitable shape in other embodiments.

The cup 16 further comprises a mount member 32 which is coupled to and extends outwardly from the perimeter wall 20. The mount member 32 has a mount surface 34 which faces away from the perimeter wall 20 and has a planar shape, though the mount surface 34 may have other suitable shapes in other embodiments to conform to various support structures. The mount member 32 extends from the upper edge 24 of the perimeter wall 20 to the base wall 18. The adhesive 38 is coupled to the mount surface 34 of the mount member 32 and is configured to secure the container 14 to a support surface.

The cup 16 further comprises a protrusion 36 which is coupled to and extends inwardly from the perimeter wall 20. The protrusion 36 is also positioned adjacent to the upper edge 24 of the perimeter wall 20. The perimeter wall 20 is integrally formed with the base wall 18, the mount member 32, and the protrusion 36.

The lid 40 is removably positionable in a closed position 42 with respect to the cup 16 in which the lid 40 covers the access opening 26. The lid 40 comprises a main body 44, which covers the access opening 26 when the lid 40 is in the closed position 42, and a handle 56 mounted to the main body 44. The main body 44 defines a plurality of airflow holes 50 which extend through a top side 46 and a bottom side 48 of the main body 44 to facilitate airflow through the main body 44. The main body 44 has a peripheral surface 52 which is complementary in shape and size to an interior surface 30 of the cup 16.

The lid 40 has a snap fit to the cup 16 when the lid 40 is positioned in the closed position 42. The peripheral surface 52 defines a groove 54 which receives the protrusion 36 of the cup 16 when the lid 40 is in the closed position 42, thereby facilitating the snap fit between the lid 40 and the cup 16. In some embodiments the lid 40 may be attached to the cup 16 such as via a hinge or the like.

A handle 56 is mounted to the top side 46 of the main body 44 and is elongated between a pair of ends 58. The handle 56 is pivotably coupled to the main body 44 at each end 58 of the pair of ends 58. The handle 56 also comprises a bendable material such that the handle 56 is freely bendable with respect to the main body 44.

In use, the tablets 12 may be positioned in the interior space 22 of the cup 16 of the container 14, and the lid 40 may be positioned in the closed position 42 to prevent the tablets 12 from spilling out of the container 14 or other objects falling into the container 14. The container 14 may be positioned on a resting surface such as atop a table 60 or may be mounted via the adhesive 38 to the support surface, which may be a vertically oriented wall surface or the like. Air is allowed to flow through the airflow apertures 28 of the cup 16 and the airflow holes 50 of the lid 40 to allow portions the tablets 12 to vaporize and exit from the container 14 in their newly vaporized form. The deodorizing material masks, dissolves, emulsifies, or otherwise neutralizes substances causing odors, and the disinfecting material inactivates or destroys microorganisms found in the surrounding atmosphere.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An air treatment system comprising:
a tablet comprising a volatile substance wherein the tablet readily vaporizes, the volatile substance comprising a material configured to treat a surrounding atmosphere; and
a container for housing the tablet, the container comprising:
a cup comprising a base wall and a perimeter wall, the perimeter wall being coupled to and extending upwardly from the base wall, the container defining an interior space surrounded by the base wall and the perimeter wall, an upper edge of the perimeter wall defining an access opening to the interior space, the cup defining a plurality of airflow apertures which extend through the cup into the interior space to facilitate airflow through the cup, the cup further comprising a mount member, the mount member being coupled to and extending outwardly from the perimeter wall, the mount member having a mount surface facing away from the perimeter wall; and
an adhesive coupled to the mount surface of the mount member, the adhesive being configured to secure the container to a support surface.

2. The air treatment system of claim 1, wherein the volatile substance comprises a deodorizing material configured to deodorize the surrounding atmosphere.

3. The air treatment system of claim 1, wherein the volatile substance comprises a disinfecting material configured to disinfect the surrounding atmosphere.

4. The air treatment system of claim 1, wherein the volatile substance is one of a plurality of volatile substances, the plurality of volatile substances including a deodorizing material configured to deodorize the surrounding atmosphere, the plurality of volatile substances including a disinfecting material configured to disinfect the surrounding atmosphere.

5. The air treatment system of claim 1, wherein the tablet is one of a plurality of tablets, each tablet of the plurality of tablets comprising a volatile substance wherein the tablet readily vaporizes, the volatile substance comprising a material configured to treat a surrounding atmosphere.

6. The air treatment system of claim 1, wherein the plurality of airflow apertures of the cup is distributed across the base wall and the perimeter wall.

7. The air treatment system of claim 1, wherein the mount surface of the mount member of the cup has a planar shape.

8. The air treatment system of claim 1, wherein the mount member of the cup extends from the upper edge of the perimeter wall to the base wall.

9. The air treatment system of claim 1, wherein the perimeter wall of the cup has a cylindrical shape.

10. The air treatment system of claim 1, wherein the perimeter wall of the cup is integrally formed with the base wall and the mount member.

11. The air treatment system of claim 1, wherein the container further comprises a lid which is removably positionable in a closed position with respect to the cup in which the lid covers the access opening, the lid comprising a main body which covers the access opening when the lid is in the closed position, the main body defining a plurality of airflow holes which extend through a top side and a bottom side of the main body to facilitate airflow through the main body, the main body having a peripheral surface which is complementary in shape and size to an interior surface of the cup.

12. The air treatment system of claim 11, wherein:
the cup of the container comprises a protrusion which is coupled to and extends inwardly from the perimeter wall, the protrusion being positioned adjacent to the upper edge of the perimeter wall; and
the peripheral surface of the main body of the lid defines a groove which receives the protrusion of the cup when the lid is in the closed position, the main body having a snap fit with the protrusion.

13. The air treatment system of claim 12, wherein the perimeter wall of the cup is integrally formed with the base wall, the mount member, and the protrusion.

14. The air treatment system of claim 11, wherein the lid further comprises a handle which is mounted to the top side of the main body.

15. The air treatment system of claim 14, wherein the handle of the lid is elongated between a pair of ends, the handle being pivotably coupled to the main body at each end of the pair of ends.

16. The air treatment system of claim 14, wherein the handle of the lid comprises a bendable material such that the handle is freely bendable with respect to the main body.

17. An air treatment system comprising:
a plurality of tablets, each tablet of the plurality of tablets comprising a plurality of volatile substances wherein the tablet readily vaporizes, the plurality of volatile substances including a deodorizing material configured to deodorize a surrounding atmosphere, the plurality of volatile substances including a disinfecting material configured to disinfect the surrounding atmosphere; and
a container for housing the plurality of tablets, the container comprising:
a cup comprising a base wall and a perimeter wall, the perimeter wall being coupled to and extending upwardly from the base wall, the container defining an interior space surrounded by the base wall and the perimeter wall, an upper edge of the perimeter wall defining an access opening to the interior space, the cup defining a plurality of airflow apertures which extend through the cup into the interior space to facilitate airflow through the cup, the plurality of airflow apertures being distributed across the base wall and the perimeter wall, the cup further comprising a mount member, the mount member being coupled to and extending outwardly from the perimeter wall, the mount member having a mount surface facing away from the perimeter wall, the mount surface having a planar shape, the mount member extending from the upper edge of the perimeter wall to the base wall, the cup comprising a protrusion which is coupled to and extends inwardly from the perimeter wall, the protrusion being positioned adjacent to the upper edge of the perimeter wall, the perimeter wall having a cylindrical shape, the perimeter wall being integrally formed with the base wall, the mount member, and the protrusion;
an adhesive coupled to the mount surface of the mount member, the adhesive being configured to secure the container to a support surface; and
a lid removably positionable in a closed position with respect to the cup in which the lid covers the access opening, the lid comprising:
a main body which covers the access opening when the lid is in the closed position, the main body defining a plurality of airflow holes which extend through a top side and a bottom side of the main body to facilitate airflow through the main body, the main body having a peripheral surface which is complementary in shape and size to an interior surface of the cup, the peripheral surface defining a groove which receives the protrusion of the cup when the lid is in the closed position, the main body having a snap fit with the protrusion; and
a handle mounted to the top side of the main body, the handle being elongated between a pair of ends, the handle being pivotably coupled to the main body at each end of the pair of ends, the handle comprising a bendable material such that the handle is freely bendable with respect to the main body.
